(19) United States
(12) Patent Application Publication
(10) Pub. No.: US 2009/0259227 A1
(43) Pub. Date: Oct. 15, 2009

(54) Reamer for Operating Implant

(76) Inventor: Sang-Hoon Ahn, Yuseong-gu (KR)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/295,667

(22) PCT Filed: Dec. 4, 2006
(86) PCT No.: PCT/KR06/05174

§ 371 Date: Oct. 1, 2008

(30) Foreign Application Priority Data
Apr. 4, 2006 (KR) 10-2006-0030450

(51) Int. Cl. A61B 17/00

(52) U.S. Cl. 606/80

(57) ABSTRACT

Disclosed therein is a reamer for operating implant, which can very usefully apply an implant operation to a patient who is short of bone quantity to the maxillary sinus, which is easier and safer than a conventional hammering method using a drill and an osteotome or a conventional operation method for transplanting a bone or planting an implant after forming a bone window on a maxillary sinus side wall, and which can reduce a treatment time period and treatment costs by minimizing the patient's pain occurring before and after the operation of the implant. A cutting part of the reamer includes: a protruding face formed on the top surface thereof in such a manner as to upwardly upheave a partial surface including an outer circumference of the cutting part of the entire top surface of the cutting part to form a stepped jaw; a depressed face formed on the top surface thereof in such a manner as to depress a partial surface of the entire top surface of the cutting part to be opposite to the protruding face according to the formation of the protruding face, the depressed face having a reverse inclination in a clockwise direction; a cut edge horizontally formed on a connected portion between the stepped jaw and the protruding face so that the cut edge evenly disperses a force while being in horizontal line contact with the mucosa even though the cut edge is in contact with the mucosa of the maxillary sinus so as to allow a dentist to perform a cutting work safely without damaging a mucosa of a maxillary sinus; and a discharge path longitudinally formed on the cutting part in such a manner as to cut an area ranging from a partial surface of the top surface of the cutting part, i.e., a predetermined portion between the protruding face and the depressed face to a portion which is slightly shorter than the lower end of the cutting part.

### TECHNICAL FIELD

The present invention relates to a reamer for operating implant, which is adapted to form a hole in a bone to plant an implant during a dental implant operation, and more particularly, to a reamer for operating implant, which can very usefully apply an implant operation to a patient who is short of bone quantity of up to the maxillary sinus, which is easier and safer than a conventional hammering method using a drill and an osteotome or a conventional operation method for transplanting a bone or planting an implant after forming a bone window on a maxillary sinus side wall, and which can reduce a treatment time period and treatment costs by minimizing the patient's pain occurring before and after the operation of the implant.

### BACKGROUND ART

In general, an implant originally means a replacement for recovering lost body tissues when tissues of the human body are lost, but means a transplantation of an artificial tooth in a dental surgical field.

The implant is the highly-advanced technology for recovering the original function of a lost tooth through the steps of planting and adhering an artificial tooth made of titanium, which does not cause rejection symptoms to the human body so as to act as a substitute for a root of the lost tooth, and then fixing the artificial tooth. A general prosthesis or a false tooth has a demerit in that it damages teeth and bone around the prosthesis or the false tooth over time, but the implant has a merit in that it does not damage teeth tissues around the implant and can be used for a long time since it does not cause a decay of teeth while providing function and shape similar to natural teeth.

In such an implant operation, it has been reported that a success rate in planting the implant in the upper molar part is lower than that in other parts. The reason for this is that a long implant cannot be planted since the upper molar part is weak in bony tissue and has the maxillary sinus.

That is, the maxillary sinus existing in the upper molar part is a space surrounded with a mucosa, and so, since it physiologically comes down and is expanded when a tooth is lost, the maxillary sinus is short of bone quantity for planting the implant thereto due to a bone absorption and the downward expansion of the maxillary sinus when the tooth is lost, whereby it is difficult to operate the implant in the upper molar part.

Meanwhile, as conventional representative operating methods performed when the patient is short of bone quantity to the maxillary sinus, there are a lateral (external) window sinus lift (sinus graft) and an internal sinus lift (sinus lift), and their operation methods will be described as follows.

First, as shown in FIG. 1 (operation is progressed in the arrow direction), when the implant is planted in the upper molar part (remaining bone quantity of less than 5 mm) which is greatly short of vertical bone quantity, the lateral window sinus lift is an operation method including the steps of cutting a maxilla side wall 101 according to a height of the remaining alveolar bone, securing a sufficient bone quantity through a grafted bone 102, and planting a long implant 103 thereto.

The operation method of the lateral window sinus lift includes a process of extracting a bone fragment from the patient's body part excepting a part which will be operated for bone graft, a process of setting a cut line on a vestibular region of a molar teeth area, a process of forming a mucoperiosteal flap, a process of windowing a face wall of the maxillary sinus after putting a fracture line using a round bur 34 when the maxillary sinus is opened, a process of lifting the face wall and the mucosa of the maxillary sinus, a process of treating a tabula in a space of the lifted maxillary sinus, a bone graft process, a suture process, and a process of planting the implant after 6 to 12 months from the operation.

The internal sinus lift is an operation method performed when the remaining bone quantity runs short a little (5˜10 mm), and as shown in FIG. 2 (the operation is progressed in the arrow direction), is a method of lifting a bone by hammering using a chisel called an osteotome, putting an autogenous bone or an artificial bone in a space formed by lifting the bone, and planting an implant in the space. That is, the internal sinus lift includes the steps of: removing the bone from a region, to which the implant will be planted, using a first drill 202 to form a hole; alternately inserting the osteotomes 201 of various diameters ranging from a smaller one to a larger one into the hole and then carefully hammering the osteotomes 201 inserted into the hole to gradually expand the hole till it gets close to the mucosa 203 of the maxillary sinus 200; fracturing only the bone without any damage of the mucosa 203 of the maxillary sinus 200; and planting the implant 205 after grafting the autogenous bone or artificial bone in a space where the bone is fractured.

However, the lateral window sinus lift has a problem in that it takes much time to ossify the grafted bone and a treatment period of time is extended since the operation is difficult and a great deal of bones are grafted.

In addition, the internal sinus lift includes a process of forming a hole to a distance as safe as the hole is not in contact with the mucosa of the maxillary sinus, namely, to a compact bone below the mucosa of the maxillary sinus, using a twist drill, a process of alternately inserting osteotomes 201 of various diameters ranging from a smaller one to a larger one into the hole and then hammering the osteotomes 201 inserted into the hole till the hole has a diameter corresponding to that of an implant, a process of fracturing the compact bone by finally hitting the osteotome when the hole proper to plant the implant is formed, a process of filling bone graft material in the hole formed in the compact bone, a process of inserting the osteotome into the hole filled with the bone graft material and smoothly hitting the inserted osteotome to thereby lift the mucosa of the maxillary sinus, and a process of planting the implant when the height of a usable bone to which the implant can be planted is secured.

However, since the twist drill used for the operation has a number of sharp blades and a peaked conical upper end, it is good to a forward cutting of the bone, but has a problem in that skilled and unskilled dentists all feel difficulty in forming the hole for planting the implant in the compact bone using the drill without any damage to the mucosa of the maxillary sinus.

That is, since the drill has the peaked conical upper end, when the upper end of the drill is in contact with the mucosa of the maxillary sinus during rotation, a vertical force is concentrated on the mucosa of the maxillary sinus, which is in point contact with the upper end of the drill, so that the mucosa of the maxillary sinus is easily ruptured and it is difficult to keep and hold the bone graft material for formation of the bone, whereby an infection rate is increased since the implant is not located inside the bone but exists inside the maxillary sinus in an exposed state. In addition, the internal sinus lift has another problem in that a force to hold and support the implant is deteriorated since the bone quantity is small.

Moreover, to fracture the compact bone below the maxillary sinus without any damage to the mucosa of the maxillary sinus by hitting the osteotome with the drill is like to break only an eggshell without any damage to the mucosa inside the eggshell. So, no matter how careful the dentist may hit the osteotome, since such operation skill can be obtained by a fine sense of the dentist's hand through abundant operation experiences, not only dentists who have no abundant operation experiences but also dentists who have abundant operation experiences had a difficulty in operation since the mucosa of the maxillary sinus is flatly ruptured when the dentist hits the osteotome a little strongly due to misadjustment of the force.

Furthermore, since the compact bone is fractured in irregular sizes and it is impossible to form the hole of a wanted type, it is impossible to obtain an initial fixation of the screw type implant into a cortical bone which is short of the remaining bone.

### DISCLOSURE OF INVENTION

### Technical Problem

Accordingly, the present invention has been made to solve the above-mentioned problems occurring in the prior art, and it is an object of the present invention to provide a reamer for operating an implant, which includes a horizontal cut edge, a protruding face and a depressed face formed on the upper end of a cutting part, thereby allowing a dentist to perform a bone graft between a mucosa of the maxillary sinus and a bone more easily and safely without any damage of the mucosa of the maxillary sinus.

Another object of the present invention is to provide a reamer for operating an implant, which can minimize a lateral window sinus lift by obtaining an initial fixation of the implant in the compact bone of the maxillary sinus when a bone quantity runs short.

### Technical Solution

To achieve the above objects, the present invention provides a reamer for operating an implant, which includes a cutting part for forming a hole for planting the implant in a bone, and a connection part having a diameter smaller than that of the cutting part and downwardly extending from the lower portion of the cutting part, wherein the cutting part includes: a protruding face formed on the top surface thereof in such a manner as to upwardly upheave a partial surface including an outer circumference of the cutting part of the entire top surface of the cutting part to form a stepped jaw; a depressed face formed on the top surface thereof in such a manner as to depress a partial surface of the entire top surface of the cutting part to be opposite to the protruding face according to the formation of the protruding face, the depressed face having a reverse inclination in a clockwise direction; a cut edge horizontally formed on a connected portion between the stepped jaw and the protruding face so that the cut edge evenly disperses a force while being in horizontal line contact with the mucosa even though the cut edge is in contact with the mucosa of the maxillary sinus so as to allow a dentist to perform a cutting work safely without damaging a mucosa of a maxillary sinus; and a discharge path longitudinally formed on the cutting part in such a manner as to cut an area ranging from a partial surface of the top surface of the cutting part, i.e., a predetermined portion between the protruding face and the depressed face to a portion which is slightly shorter than the lower end of the cutting part.

In addition, the upper end portion of the cutting part has a tapered portion formed on the outer periphery thereof.

Moreover, the cut edge is formed horizontally when it is viewed from a side.

Furthermore, the protruding face has an inclined surface inclined downwardly while forming an acute angle with the cut edge as it goes toward the right with respect to the stepped jaw.

Additionally, the depressed face has a reversely inclined surface formed in the clockwise direction and downwardly inclined while forming an acute angle with the cut edge as it goes toward the front with respect to the stepped jaw, whereby the depressed face serves to upwardly lift bone fragments when the reamer is rotated forwardly (in the clockwise direction).

Moreover, a screw portion is formed on the outer periphery of the lower portion of the cutting part and connected with the discharge path, whereby waste matters of the bone generated when the reamer is rotated forwardly and backwardly are lifted upwardly or discharged downwardly along the screw portion.

Furthermore, the screw portion is constructed of a left-hand screw type, whereby the waste matters of the bone are lifted upwardly toward the mucosa of the maxillary sinus along the left-hand screw portion when the reamer is rotated in the clockwise direction, and discharged downwardly along the left-hand screw portion when the reamer is rotated in the counterclockwise direction.

### BRIEF DESCRIPTION OF THE DRAWINGS

FIG. 1 is a view showing an operation process of a lateral window sinus lift according to the prior art.

FIG. 2 is a view showing an operation process of an internal sinus lift according to the prior art.

FIG. 3 is a perspective view of a reamer according to a preferred embodiment of the present invention.

FIG. 4 is a partially enlarged view of the reamer according to the present invention.

FIG. 5 is a view showing an implant operating process of the reamer according to the present invention.

FIG. 6 is a comparative view showing a state where a drill and the reamer are used according to the present invention.

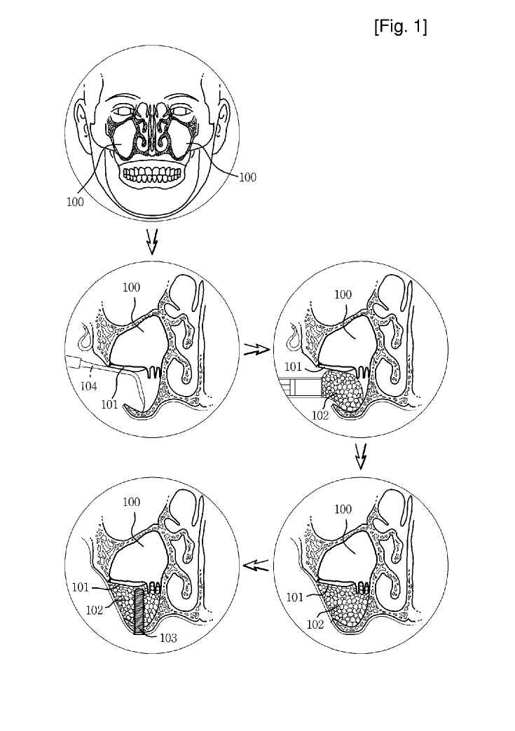

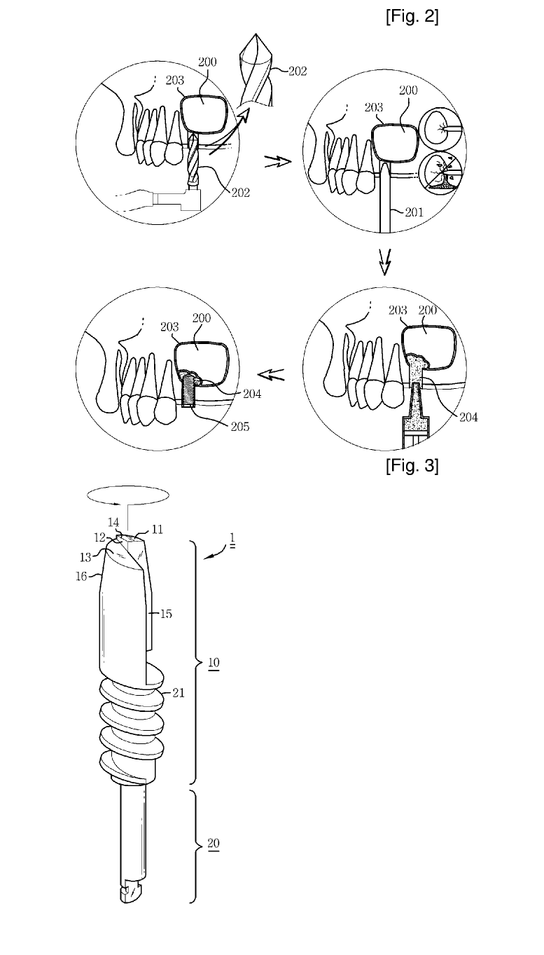

### EXPLANATION ON ESSENTIAL REFERENCE NUMERALS IN DRAWINGS

1: reamer 10: cutting part 11: protruding face

12: stepped jaw 13: depressed face 14: cut edge

15: discharge path 16: tapered portion

20: connection portion 21: screw portion

30: maxillary sinus 31: mucosa

32: bone 33: hole 34: implant

35: compact bone 36: grafted bone 37: drill

38: bone fragment

### MODE FOR THE INVENTION

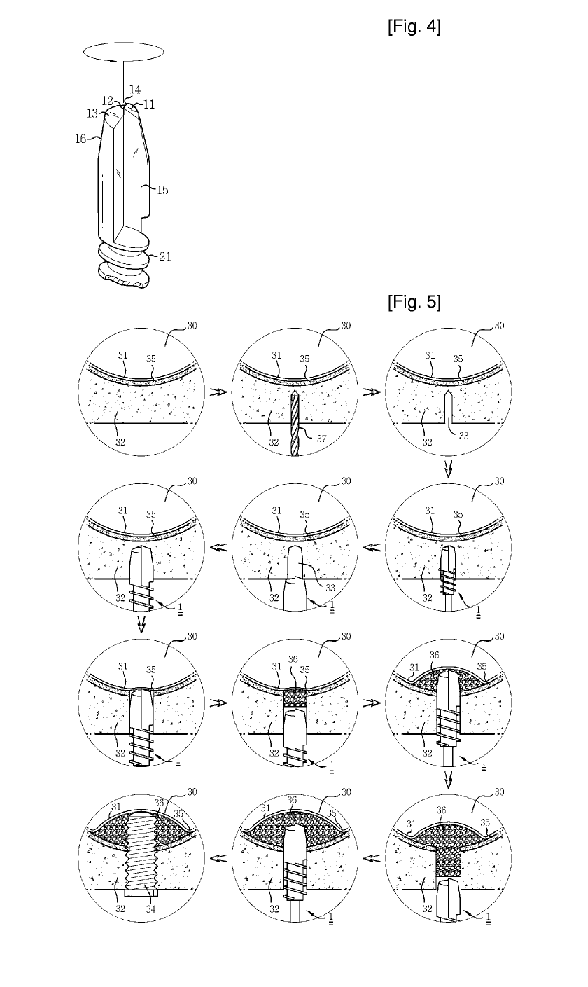

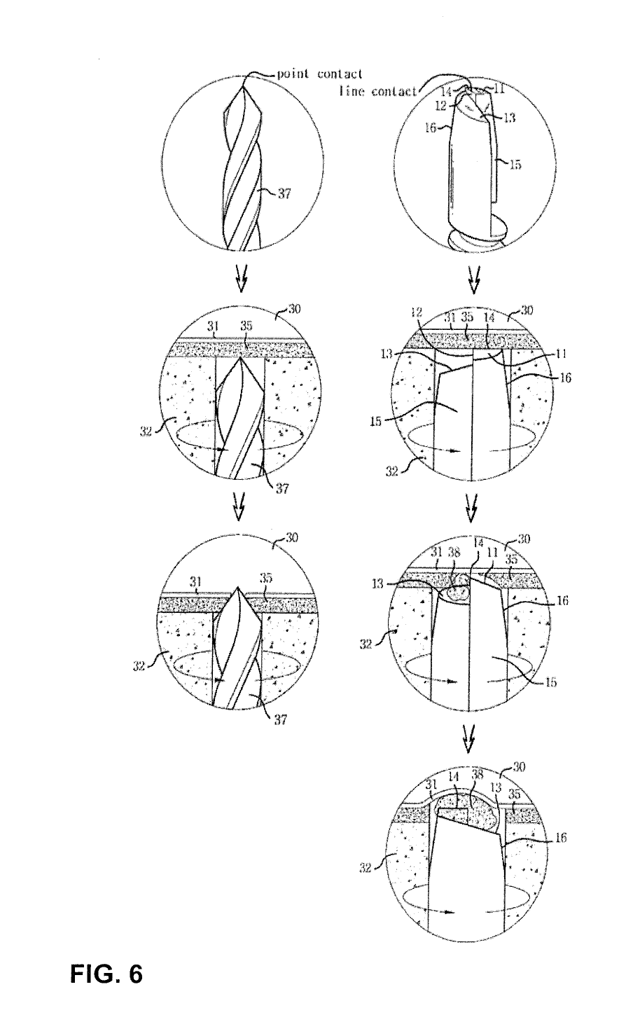

FIG. 3 is a perspective view of a reamer according to a preferred embodiment of the present invention, FIG. 4 is a partially enlarged view of the reamer according to the present invention, FIG. 5 is a view showing an implant operating process of the reamer according to the present invention, and FIG. 6 is a comparative view showing a state where a drill and the reamer are used according to the present invention. Hereinafter, reference will now be made in detail to the preferred embodiments of the present invention, examples of which are illustrated in the accompanying drawings.

As shown in FIGS. 3 and 4, the reamer 1 according to the present invention includes a cutting part 10 for forming a hole 33 for planting an implant 34 in a bone 32, and a connection part 20 extending from the bottom side of the cutting part 10 and having a diameter smaller than that of the cutting part 10.

The cutting part 10 is in the form of a cylinder having a predetermined diameter, and the upper portion of the cutting part 10 has a tapered portion 16 or is formed in a straight type without any tapered portion 16.

That is, to operate the implant 34, a number of the reamers 1 of various diameters are used as a reamer set, and in this instance, the reamer 1 of the smallest diameter has the cutting part 10 formed in the straight type without any tapered portion 16 but the other reamers respectively have the cutting parts 10 on each of which the tapered portion 16 is formed.

Moreover, the cutting part 10 has a protruding face 11 formed on the top surface thereof in such a manner as to upwardly upheave a partial surface including an outer circumference of the entire top surface of the cutting part 10 to form a stepped jaw 12, and a cut edge 14 naturally formed on a connected portion between the stepped jaw 12 and the protruding face 11.

In this instance, the protruding face 11 is formed on the top surface of the cutting part 10 in such a manner as to upheave a portion corresponding to a fourth of the front side of the cutting part 10, and more concretely, a portion corresponding to a half upper side of the right side semicircle from the stepped jaw 12 when it is viewed from a plane.

In addition, the stepped jaw 12 is naturally formed on the cutting part 10 according to the formation of the protruding face 11, and is a standard for dividing the protruding face 11 and a depressed face 13 formed to be opposite to the protruding face 11.

Since the cut edge 14 is horizontally formed when it is viewed from a side, when it cuts the bone 32 for operating the implant 34, the cut edge 14 is in horizontal line contact with a mucosa 31 of a maxillary sinus 30 even though it touches the mucosa 31 of the maxillary sinus 30, whereby the reamer 1 can cut only the bone safely without damaging the mucosa 31 of the maxillary sinus 30 since the whole cut edge 14 horizontally applies a force to the mucosa 31 without vertically concentrating the force on one position.

Furthermore, the protruding face 11 is downwardly inclined while forming an acute angle with the cut edge 14 as it goes toward the right with respect to the stepped jaw 12, and so, the cutting of the bone 32 is achieved only by the cut edge 14.

Meanwhile, the depressed face 13 is naturally formed on the top surface of the cutting part 10 to be opposite to the protruding face according to the formation of the protruding face. The depressed face 13 is downwardly inclined while forming an acute angle with the cut edge 14 as it goes toward the front with the respect to the stepped jaw 12.

The depressed face 13 corresponds to a left side semicircle with the respect to the stepped jaw 12 when it is viewed from a plane. Bone fragments 38 cut by the cut edge 14 are gathered on the depressed face 13 and have pressure, and in this instance, such pressure acts as pressure to direct the mucosa 31 of the upper maxillary sinus 30 when the reamer 1 is rotated in the clockwise direction by a reverse inclination of the inclinedly depressed face 13.

Therefore, when the reamer 1 is rotated, since the cut edge 14 is not in contact with the mucosa 31 of the maxillary sinus 30 but the bone fragments 38 accumulated on the depressed face 13 are in contact with the mucosa 31 of the maxillary sinus 30, a dentist can perform the operation of the implant safely without damaging the mucosa 31.

The cutting part 10 has a discharge path 15 formed on a side thereof to discharge waste matters of the bone 32, which are cut by the cut edge 14, along the discharge path 15.

The discharge path 15 is a groove formed on the side of the cutting part 10 in such a manner as to cut a half lower side of the right side semicircle when it is viewed from a plane with respect to the stepped jaw 12, and is formed from the upper end of the cutting part 10 to a portion which is slightly shorter than the lower end of the cutting part 10.

Meanwhile, a screw portion 21 is formed on the lower portion of the cutting part 10, if necessary.

That is, the screw portion 21 formed on the outer circumference of the lower portion of the cutting part 10 is connected with the discharge path 15 and serves to upwardly lift the waste matters of the bone 32 generated according to the forward rotation and reverse rotation of the reamer 1 through the discharge path 15 along the screw portion 21 or discharge them through the discharge path 15 along the screw portion 21, and so, the dentist can operate the implant more conveniently.

In this instance, the screw portion 21 formed on the cutting part 10 is constructed of a left-hand screw type. So, when the reamer 1 rotates in the clockwise direction (in an arrow direction in FIG. 3), the waste matters of the bone 32 are upwardly lifted toward the mucosa 31 of the maxillary sinus 30 through the discharge path 15 along the screw portion 21, but when the reamer 1 rotates in the counterclockwise direction, the waste matters of the bone 32 are discharged downwardly along the screw portion 21.

The connection part 20 is formed on the lower side of the cutting part 10, and has a diameter smaller than that of the cutting part 10.

The reamer 1 according to the present invention evenly disperses a force in parallel with the mucosa 31 without vertically concentrating the force on the mucosa 31 even though the cut edge 14 is in contact with the mucosa 31 of the maxillary sinus 30 since the cut edge 14 is horizontally formed on the connected portion between the stepped jaw 12 and the protruding face 11, whereby the dentist can perform a cutting work safely without damaging the mucosa 31 of the maxillary sinus 30. Hereinafter, referring to the drawings, a process of operating the implant 34 using the reamer 1 of the present invention will be described as follows.

As shown in FIG. 5 (operation is progressed in the arrow direction), first, the dentist measures a distance to the maxillary sinus 30 on an autoradiograph, and then, to rapidly cut a compact bone 35, cuts the bone 32 to a safe portion which does not damage the mucosa 31 of the maxillary sinus 30, using a general drill 37 for operating the implant 34 to form the hole 33.

After that, the dentist inserts the reamers of various diameters ranging from a smaller one to a larger one into the hole 33 to expand and deepen the hole 33 till the diameter of the hole 33 becomes a diameter suitable for planting the implant 34. When the hole 33 suitable for planting the implant 34 is formed, the dentist cut the compact bone 35 with the final reamer 1 and lifts the mucosa 31 of the maxillary sinus 30.

When the compact bone 35 is cut by the reamer 1, the hole 33 is filled with mixture of an autogenous bone, which is extracted from a patient, and a synthetic bone, and in the above state, the mucosa 31 of the maxillary sinus 30 is lifted in such a manner as to upwardly lift the bone while the reamer 1 is rotated and advances forwardly.

The above operation is repeatedly performed till a grafted bone 36 sufficient for planting the implant 34 is secured.

After that, when the grafted bone 36 is in a state where the implant 34 can be planted in the grafted bone 36 after predetermined time period, the implant 34 is planted and fixed in the hole 33 formed in the compact bone 35 of the maxillary sinus 30, and thereby, the operation is completed.

As described above, since the prior art osteotome cannot fracture the bone in a wanted exact size, it is not of help to fix the implant. However, since the reamer 1 according to the present invention can form the hole 33 of a wanted size suitable for fixing the implant 34 thereto when the bone is cut, it makes an initial fixation of the implant 34 possible even in case where bone quantity is small. In addition, even in case where bone quantity is as small as the lateral window sinus lift is needed, the reamer 1 according to the present invention can be used for operating the implant 34.

Meanwhile, FIG. 6 is a comparative view showing states when the drill and the reamer are used according to the present invention. Referring to FIG. 6, operation processes (operation is progressed in the arrow direction) will be described as follows.

As shown in FIG. 6, since the prior art drill 37 has the peaked and sharp upper end, when the peaked upper end is in contact with the mucosa 31 of the maxillary sinus 30, there is a great possibility that the peaked upper end may damage the mucosa 31 since it concentrates a vertical force only on one position of the mucosa 31. However, the reamer 1 according to the present invention can allow the dentist to operate the implant safely without damaging the mucosa 31 of the maxillary sinus 30 since the reamer 1 evenly disperses the force in parallel with the mucosa 31 without concentrating the vertical force on the mucosa 31 even though the cut edge 14 is in contact with the mucosa 31 of the maxillary sinus 30.

In addition, the reamer 1 according to the present invention can allow the dentist to operate the implant safely without damaging the mucosa 31 of the maxillary sinus 30 since the bone fragments 38 accumulated on the depressed face 13 is in contact with the mucosa 31 of the maxillary sinus 30 before the cut edge 14 when the reamer 1 is rotated.

That is, as shown in FIG. 6, when the reamer 1 is rotated in the clockwise direction (in the arrow direction in the drawing), the bone fragments 38 cut by the cut edge 14 are accumulated on the depressed face 13. In this instance, since the depressed face 13 has a reversely inclined surface to the rotational direction of the reamer 1, namely, the clockwise direction, the bone fragments 38 accumulated on the depressed face 13 are lifted up toward the mucosa 31 while the reamer 1 is rotated in the clockwise direction so that the bone fragments 38 are first in contact with the mucosa 31. In this instance, since the cut edge 14 is not in contact with the mucosa 31 but the bone fragments 38 are in contact with the mucosa 31, the dentist can operate the implant safer without damaging the mucosa 31 of the maxillary sinus 30.

Of course, the bone fragments 38 accumulated on the depressed face 13 is not continuously accumulated but a part of the accumulated bone fragments 38 are discharged through the discharge path 15 when pressure exceeds a predetermined limit.

If the reamer 1 is rotated in counterclockwise direction (in the opposite direction of the arrow indicated in FIG. 6), on the contrary, cutting force on the bone is increased, and the cut bone fragments 38 are naturally discharged through the discharge path 15 while sliding along the inclined surface of the depressed face 13.

### INDUSTRIAL APPLICABILITY

As described above, the reamer according to the present invention has the following advantages.

1. The reamer for operating the implant according to the present invention can allow the dentist to operate the implant very safely since it can structurally minimize a damage of the mucosa of the maxillary sinus.

2. Conventionally, the hammering method using the osteotome is unavoidably applied to the patient who is short of bone quantity to the maxillary sinus. However, the reamer according to the present invention can minimize the patient's fear and pain felt before and after the operation since the reamer adopts an operation manner of perforating the hole in the bone.

3. The reamer according to the present invention can allow the dentist to easily plant the implant in the optimum place safely even in case where a structure, such as a partition wall or a side wall, exists inside the maxillary sinus.

4. The reamer according to the present invention can form the exact hole similar to the diameter of the implant, which will be planted, in the cortical bone since the reamer adopts the operation manner not of breaking the cortical bone of the bottom of the maxillary sinus but of forming the hole, and so, provide an environment advantageous for ossification since the implant is united to the hole of the cortical bone of the maxillary sinus very exactly and stably.

5. The reamer according to the present invention has an advantage in that the dentist may not perform a hard operation for forming a bone window on a side wall since it can provide a good initial fixation of the implant even in a condition of the small bone quantity of the maxillary sinus.

6. The reamer according to the present invention allows the dentist to easily perform a bone graft in the maxillary sinus as much as it is necessary without perforating or rupturing the mucosa of the maxillary sinus by the reamer's forward and backward rotations.

7. The reamer according to the present invention can reduce bone quantity used for operation since it allows the dentist to graft the bone in the mucosa of the maxillary sinus as much as the bone quantity is needed exactly, and greatly reduce a treatment period time by reducing an ossification period of the grafted bone since this method according to the present invention uses the bone smaller than the prior art operation method.

8. The reamer according to the present invention can provide a reduction effect of medical expenses by reducing a use of an expensive substitute bone since it can expand the hole and extract the patient's fresh bone.

1. A reamer for operating an implant, which comprises a cutting part for forming a hole for planting the implant in a bone, and a connection part having a diameter smaller than that of the cutting part and downwardly extending from the lower portion of the cutting part,
wherein the cutting part comprises:
a protruding face formed on the top surface thereof in such a manner as to upwardly upheave a partial surface including an outer circumference of the entire top surface of the cutting part to form a stepped jaw;
a depressed face formed on the top surface thereof in such a manner as to depress a partial surface of the entire top surface of the cutting part to be opposite to the protruding face according to the formation of the protruding face, the depressed face having a reverse inclination in a clockwise direction;
a cut edge horizontally formed on a connected portion between the stepped jaw and the protruding face so that the cut edge evenly disperses a force while being in horizontal line contact with a mucosa even though the cut edge is in contact with the mucosa of the maxillary sinus so as to allow a dentist to perform a cutting work safely without damaging the mucosa of the maxillary sinus; and
a discharge path longitudinally formed on the cutting part in such a manner as to cut an area ranging from a partial surface of the top surface of the cutting part, the discharge path extending from a predetermined portion between the protruding face and the depressed face to a portion which is slightly shorter than the lower end of the cutting part.

2. The reamer for operating the implant according to claim 1, wherein the upper end portion of the cutting part has a tapered portion formed on the outer periphery thereof.

3. The reamer for operating the implant according to claim 1, wherein the cut edge is formed horizontally when it is viewed from a side.

4. The reamer for operating the implant according to claim 1, wherein the protruding face has an inclined surface inclined downwardly while forming an acute angle with the cut edge as it goes toward the right with respect to the stepped jaw.

5. The reamer for operating the implant according to claim 1, wherein the depressed face has a reversely inclined surface formed in the clockwise direction and downwardly inclined while forming an acute angle with the cut edge as it goes toward the front with respect to the stepped jaw, whereby the depressed face serves to upwardly lift bone fragments when the reamer is rotated forwardly (in the clockwise direction).

6. The reamer for operating the implant according to claim 1, wherein a screw portion is formed on the outer periphery of the lower portion of the cutting part and connected with the discharge path, whereby waste matters of the bone generated when the reamer is rotated forwardly and backwardly are lifted upwardly or discharged downwardly along the screw portion.

7. The reamer for operating the implant according to claim 1, wherein the screw portion is constructed of a left-hand screw type, whereby the waste matters of the bone are lifted upwardly toward the mucosa of the maxillary sinus along the left-hand screw portion when the reamer is rotated in the clockwise direction, and discharged downwardly along the left-hand screw portion when the reamer is rotated in the counterclockwise direction.
